(19) United States
(12) Patent Application Publication
(10) Pub. No.: US 2023/0135701 A1
(43) Pub. Date: May 4, 2023

(54) DRUG DISCOVERY USING A PSEUDO CONCENTRATION-RESPONSE CURVE

(71) Applicant: ARCTORIS LIMITED, Abingdon (GB)

(72) Inventors: Daniel A. Thomas, Oxford (GB); Hester M. Sheehan, London (GB); Nancy O. Johnson, Oxford (GB)

(21) Appl. No.: 17/452,884

(22) Filed: Oct. 29, 2021

(51) Int. Cl. G01N 33/50

(52) U.S. Cl. CPC G01N 33/502

(57) ABSTRACT

A method is provided for screening a compound relative to a target biological component. The method includes determining an estimated half maximal inhibitory concentration (IC₅₀) value for the compound relative to the target biological component from three concentrations of the compound. This includes testing the three concentrations on the target biological component to obtain three response measurements of the compound in inhibiting a biological function of the target. A pseudo concentration-response curve (CRC) is constructed from the three concentrations and three response measurements, and the estimated IC₅₀ value is determined from the pseudo CRC. The method includes determining a plurality of concentrations of the compound from the estimated IC₅₀ value, and testing the plurality of concentrations of the compound on the target biological component to obtain a plurality of response measurements of the compound. And a CRC is constructed from the plurality of concentrations and the plurality of response measurements.

### TECHNOLOGICAL FIELD

The present disclosure relates generally to drug discovery and, in particular, to drug discovery using a pseudo concentration-response curve.

### BACKGROUND

In the pharmaceutical industry, single-shot screening remains an effective method of identifying hit molecules within large compound sets. Hit molecules are those that have a specific desired activity against a specific biological target of interest. The single-shot approach takes a single concentration of a molecule and tests it once to see what effect, if any, it has on a specific biological target. Serial dilution, which describes the stepwise dilution of a molecule, is then routinely employed to generate concentration-response curves (FIG. 1). These curves are in turn used to calculate half maximal inhibitory concentration (IC₅₀) values used in the optimization of hit molecules.

Despite its popularity, single-shot screening has several unsatisfactory aspects. Single-shot screens require a high level of sensitivity to enable relatively weak compounds to be detected and must be robust enough to minimize the rates of false negative and false positive compounds. While false positives can be ruled out at a later stage, by their very nature, false negatives will be missed resulting in the loss of potentially critical data.

The IC₅₀ value is widely used in the pharmaceutical industry to assess a drug's potency. In this regard, IC₅₀ is a context-dependent quantitative measure (typically expressed in molar concentration), which indicates how much of a particular molecule (e.g., drug) is needed to inhibit a given biological process or biological component by 50% in vitro (FIG. 2). The IC₅₀ values are obtained by fitting normalized response data from a target across a range of concentrations of a test molecule using a four-parameter logistical equation. The resulting concentration-response curves from these experiments are widely used to compare the relative inhibitor potencies of many different molecules against the same target, tested under a set of controlled conditions.

Despite their convenience and popularity, the context-dependent nature of IC₅₀ values is subject to change with changing assay conditions. In the case of a competitive or uncompetitive inhibitor, the observed IC₅₀ value will depend on the concentration of substrate present in the assay relative to the Michaelis-Menten Constant (Kₘ) for a specific substrate; in other words, concentration of substrate necessary to generate half the maximal enzyme activity.

Serial dilutions are frequently used in biological research. They underpin many assay development processes and are critical in enabling the evaluation of drug potency and efficacy. Typically, the dilution factor remains constant at each step, creating a geometric progression of concentration in a logarithmic fashion and allowing concentration response curves for each test molecule to be generated. While they can access wide concentration ranges enabling a single process to be used to test a wide variety of potencies, the serial dilution methodology faces two major challenges: process variation and error propagation.

Process variation can be eliminated by standardizing the serial dilution method and utilizing automated liquid-handling solutions to ensure consistent process execution. Error propagation across a concentration series arises due to the accumulation of variability from several sources including adsorption, inadequate mixing and transfer inaccuracies. The impact of this error propagation is most acutely observed in drug potency (IC₅₀) and Hill slope parameters, both of which are critical for the development of novel molecules.

It would therefore be desirable to have a system and method that takes into account at least some of the issues discussed above, as well as other possible issues.

### BRIEF SUMMARY

Example implementations of the present disclosure are directed to drug discovery and, in particular, to drug discovery using a pseudo concentration-response curve (pseudo-CRC). This pseudo-CRC technique is an adaptation of single-shot screening that generates an initial potency prediction paired with a targeted full curve approach to achieve a more precise IC₅₀ value than often found in conventional techniques.

As introduced above, the Hill slope quantifies the steepness of a dose-response curve: a steeper curve means a greater Hill slope, while potency is described as the amount of a drug needed to elicit 50% of the maximal effect of that drug. The pseudo-CRC technique of example implementations aims to improve the confidence in and precision of these parameters by replacing the serial dilution method with direct, focused digital titrations and optimizing each of the associated processes by which they are determined.

The present disclosure thus includes, without limitation, the following example implementations.

Some example implementations provide a system for screening a compound relative to a target biological component, the system comprising a computer including: a memory configured to store computer-readable program code; and processing circuitry configured to access the memory, and execute the computer-readable program code to cause the computer to at least: determine an estimated half maximal inhibitory concentration (IC₅₀) value for the compound relative to the target biological component from three concentrations of the compound, including the computer caused to: test the three concentrations on the target biological component to obtain three response measurements of the compound in inhibiting a biological function of the target; construct a pseudo concentration-response curve (CRC) from the three concentrations and the three response measurements, the pseudo CRC constructed as a sigmoidal curve to which the three concentrations and the three response measurements are fit using the Hill equation; and determine the estimated IC₅₀ value from the pseudo CRC; determine a plurality of concentrations of the compound from the estimated IC₅₀ value, the plurality of concentrations thereby specific to the compound relative to the target; test the plurality of concentrations of the compound on the target biological component to obtain a plurality of response measurements of the compound; and construct a CRC from the plurality of concentrations and the plurality of response measurements.

Some example implementations provide a method of screening a compound relative to a target biological component, the method comprising: determining an estimated half maximal inhibitory concentration (IC₅₀) value for the compound relative to the target biological component from three concentrations of the compound, including: testing the three concentrations on the target biological component to obtain three response measurements of the compound in inhibiting a biological function of the target; constructing a pseudo concentration-response curve (CRC) from the three concentrations and the three response measurements, the pseudo CRC constructed as a sigmoidal curve to which the three concentrations and the three response measurements are fit using the Hill equation; and determining the estimated IC₅₀ value from the pseudo CRC; determining a plurality of concentrations of the compound from the estimated IC₅₀ value, the plurality of concentrations thereby specific to the compound relative to the target; testing the plurality of concentrations of the compound on the target biological component to obtain a plurality of response measurements of the compound; and constructing a CRC from the plurality of concentrations and the plurality of response measurements.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURE(S)

Having thus described example implementations of the disclosure in general terms, reference will now be made to the accompanying figures, which are not necessarily drawn to scale, and wherein:

FIG. 1 illustrates a generic serially-spaced, eleven-point concentration-response curve (CRC);

FIG. 2 illustrates a CRC highlighting how the half maximal inhibitory concentration (IC₅₀) value is defined, according to some example implementations;

FIG. 3 illustrates a system for screening a compound relative to a target biological component, according to some example implementations;

FIG. 4 illustrates a pseudo CRC with three points for the three concentrations and response measurements, according to some example implementations;

FIG. 5 illustrates an example of a CRC constructed according to some example implementations;

FIG. 6 is a flow diagram illustrating various steps in a method of screening a compound, according to some example implementations;

FIGS. 7A, 7B, 7C, 7D, 7E, 7F, 7G, 7H, 7I and 7J are flowcharts illustrating various steps in a method of screening a compound relative to a target biological component, according to various example implementations; and

FIG. 8 illustrates an apparatus according to some example implementations.

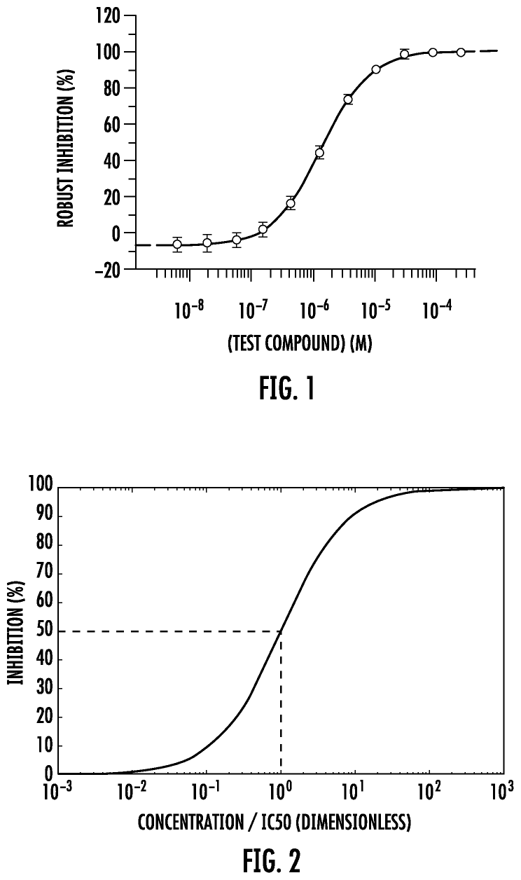

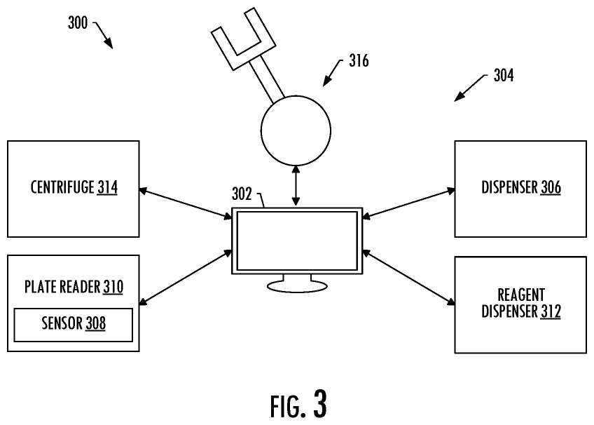

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably.

During the early stages of drug development, the conventional approach to testing compounds is to run standard eleven-point, serially-spaced concentration-response curves (CRCs) for all compounds (FIG. 1). Generally, stock solutions of compounds are made up at 10 millimolar (mM) in 100% dimethyl sulfoxide (DMSO), typically run from an uppermost concentration of 100 micromolar (μM) (limiting the DMSO concentration to 1%) and then serially diluted between 2- and 4-fold to test a broad range of different concentrations.

Example implementations of the present disclosure provide a pseudo concentration-response curve (pseudo-CRC) technique for drug discovery that differs from this traditional method. In some example implementations, the pseudo-CRC technique allows the generation of predicted potencies using initial three-point CRCs for triangulation. This technique, using three instead of eleven concentration points, allows an estimate of the half-maximal inhibitory concentration (IC₅₀) to be calculated that can subsequently be used to create a bespoke targeted eleven-point concentration range for each compound tested. Although described in the context of an eleven-point concentration range, it should be understood that example implementations may be equally applicable to concentration ranges with fewer than eleven points, as well as those with greater than eleven points.

FIG. 3 illustrates a system 300 for screening a compound relative to a target biological component, according to some example implementations. As shown, the system includes a computer 302; and in this regard, a “computer” is generally a machine that is programmable to programmed to perform functions or operations. The system also includes laboratory equipment 304. In some examples, the computer and laboratory equipment may be located in a laboratory, although it should be understood that the computer may be located outside the laboratory. It should also be understood that the laboratory equipment may be located in one laboratory, or distributed across multiple laboratories. Even further, the system may include additional or alternative components than those speifically shown and described herein.

The laboratory equipment 304 may include one or more of each of a number of different types of equipment, and that this equipment may be automated or manually operated. In this regard, the laboratory equipment may include a dispenser 306, and a sensor 308 that may be part of a plate reader 310. Examples of suitable dispensers in particular include any of a number of different non-contact dispensers sucha as a digital dispenser, acoustic dispenser, piezo dispenser, or positive displacement dispenser. An even more particular example of a non-contact dispenser is the D300e Digital Dispenser from Tecan Group Ltd., Mannedorf, Switzerland. Examples of a suitable plate reader include the CLARIOstar® and PHERAstar® from BMG LABTECH, Ortenberg, Germany.

Other examples of suitable laboratory equipment include a reagent dispenser 312, a centrifuge 314 and the like. More particular examples include the dragonfly® discovery dispenser from SPT Labtech, Cambridge, UK, and the Thermo Megafuge™ 40R plate centrifuge from Thermo Fisher Scientific, Waltham, Mass. And as also shown, in some examples, the system includes at least one robot 316, which may be any of a number of different machines configured to automatically carry out one or more actions to automate operations performed according to example implementations of the present disclosure, such as under control of the computer 304. Examples of suitable robots include those typically involved in laboratory robotics.

According to some example implementations, screening a compound relative to a target biological component includes the computer 302 configured to determine an estimated IC₅₀ value for the compound relative to the target biological component from three concentrations of the compound, using a pseudo CRC. As explained above, given the pseudo CRC that uses three instead of eleven concentration points, the estimated IC₅₀ value can be used to create a bespoke targeted concentration range (e.g., eleven-point range) for the compound. This approach means that the targeted concentration range is tighter than what would be observed with a conventional, serially-spaced concentration range.

In some examples, the three concentrations have a dilution factor that is a defined multiplier for the three concentrations. The three concentrations may also include a lowermost concentration, an intermediate concentration that is a multiple of the lowermost concentration for the defined multiplier, and an uppermost concentration that is a multiple of the intermediate concentration for the defined multiplier. In a more particular example, the dilution factor is 10, the lowermost concentration is 100 nanomolar, the intermediate concentration is 1 micromolar, and the uppermost concentration is 10 micromolar. And in another example, the dilution factor is 40, the lowermost concentration is 25 nanomolar, the intermediate concentration is 1 micromolar, and the uppermost concentration is 40 micromolar.

The computer 302 configured to determine the estimated IC₅₀ value from the three concentrations of the compound may include the computer configured to test the three concentrations on the target biological component to obtain three response measurements of the compound in inhibiting a biological function of the target. This may include the computer configured to control the robot 316 to prepare for an assay in which the dispenser is used to dispense (e.g., inkjet print) the three concentrations of the compound into wells of an assay plate. The assay may then be performed with the target biological component in which the three response measurements are obtained from the sensor 308 as the compound interacts with the target.

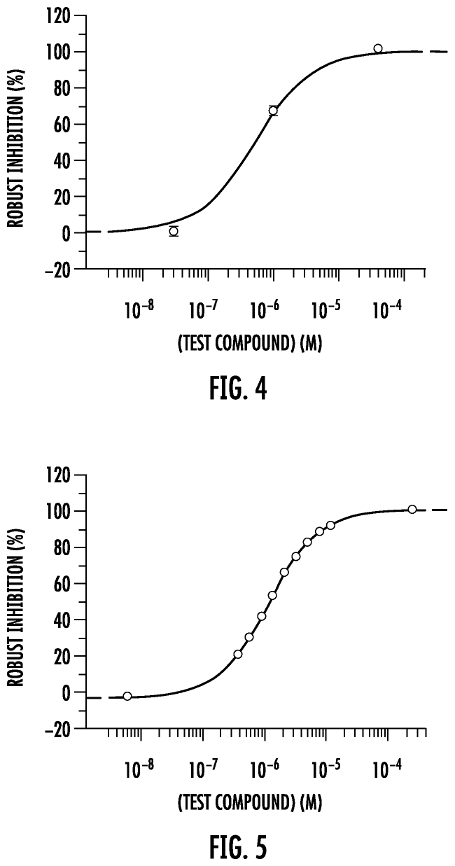

The computer 302 is configured to construct a pseudo CRC from the three concentrations and the three response measurements. In this regard, the pseudo CRC is constructed as a sigmoidal curve to which the three concentrations and the three response measurements are fit using the Hill equation. FIG. 4 illustrates one example of a suitable pseudo CRC with three points for the three concentrations and response measurements. And the computer is configured to determine the estimated IC₅₀ value from the pseudo CRC. This may include the computer configued to determine an inflection point of the pseudo CRC that corresponds to the estimated IC₅₀ value.

The Hill equation is a four-parameter logistic nonlinear model with parameters of the sigmoidal curve including a minimum asymptote, a maximum asymptote, an inflection point that corresponds to the IC₅₀, and a Hill slope. In some examples, the computer 302 configured to construct the pseudo CRC includes the computer configured to perform a regression analysis of the four-parameter logistic nonlinear model in which the minimum asymptote, the maximum asymptote and the Hill slope are constrained to predetermined values, and the inflection point is unconstrained. In the pseudo CRC shown in FIG. 4, the minimum asymptote is constrained to 0, the maximum asymptote is constrained to 100, and the Hill slope is consrained to 1.

The computer 302 is configured to determine a plurality of concentrations (e.g., eleven concentrations) of the compound from the estimated IC₅₀ value, and the plurality of concentrations are thereby specific to the compound relative to the target. In this regard, the plurality of concentrations may cover a range that is targeted based on the estimated IC₅₀ value, as opposed to the conventional, serially-spaced concentration range. The plurality of concentrations may be determined in a number of different manners. In some examples, uppermost and lowermost ones of the plurality of concentrations are determined based on maximum and minimum concentrations the dispenser 306 is designed to dispense.

Those of the plurality of concentrations between the uppermost and lowermost concentrations may be determined in a manner that includes the computer 302 configured to define a target titration factor as a dilution multiplier to achieve a defined multiplier across the concentrations, with the defined multiplier indicated by a dilution factor for the three concentrations. The computer may define upper and lower IC₅₀ limits based on the estimated IC₅₀ value, and the target titration factor. And the computer may determine a midpoint one of the concentrations that is the estimated IC₅₀ value when the estimated IC₅₀ value is between the upper and lower IC₅₀ limits, and a value that is equidistant between the upper and lower IC₅₀ limits when the estimated IC₅₀ value is not between the upper and lower IC₅₀ limits.

The computer 302 may determine those of the concentrations above the midpoint from multiplication of the midpoint and exponentiations of the target titration factor by respective exponents. The computer may determine at least some of those of the concentrations below the midpoint from division of the midpoint by exponentiations of the target titration factor by respective exponents. Now consider a next lowermost one of the concentrations that is immediately between the lowermost one of the plurality of concentrations and a second next lowermost one of the concentrations, the computer may do something different. For this next lowermost concentration, the computer may divide the midpoint by an exponentiation of the target titration factor when the estimated IC₅₀ value is less than an upper concentration limit, or take the value that is equidistant between the lowermost and the second next lowermost ones of the plurality of concentrations when the estimated IC₅₀ value is greater than an upper concentration limit.

Regardless of the exact manner by which the computer 302 is configured to determine the plurality of concentrations, the computer is configured to test the plurality of concentrations of the compound on the target biological component to obtain a plurality of response measurements of the compound, such as in a manner similar to that described above for the three concentrations. That is, the computer may control the robot 316 to prepare for an assay in which the dispenser is used to disepnse (e.g., inkjet print) the plurality of concentrations of the compound into wells of an assay plate (a second assay plate). The assay may then be performed with the target biological component in which the plurality of response measurements are obtained from the sensor 308 as the compound interacts with the target.

The computer 302 is configured to construct a CRC from the plurality of concentrations and the plurality of response measurements; and in some examples, the CRC is constructed as a second sigmoidal curve to which the plurality of concentrations and the plurality of response measurements are fit using the Hill equation. In some examples, the computer may construct the CRC from a regression analysis of the four-parameter logistic nonlinear model in which the minimum asymptote, the maximum asymptote, the Hill slope and the inflection point are all unconstrained. And again with reference to the three concentrations as described, the three concentrations may be selected to cover a range of inhibition measurements from 13.65% to 86.25% when the plurality of concentrations are tested, and span a Hill slope of the CRC when constructed from the plurality of concentrations and the plurality of response measurements.

FIG. 5 illustrates an example of a CRC constructed according to example implementations of the present disclosure. As explained above, the targeted approach to determining the plurality of concentrations means that the range of the plurality of concentrations is tighter than those observed with conventional serially-spaced concentration ranges. And as also shown, the majority of the data points in the CRC may be clustered between the inflection points for both asymptotes and span the slope of the dose response curve, that portion of the graph that allows for maximal data value. The CRC from this approach is ultimately more informative about compound potency, Hill slope and behavior. Increased data precision coupled with equivalent or better statistical confidence can be achieved from a single, targeted CRC, which may eliminate the need to complete traditional serially-spaced replicates in duplicate or triplicate. The approach may therefore be overall more cost effective as more valuable data may be obtained with fewer experiments.

In some examples, the computer 302 may be further configured to determine an IC₅₀ value for the compound relative to the target biological component, from the CRC. This may include the computer configured to determine an inflection point of the CRC that corresponds to the IC₅₀ value, in a manner similar to that described above for the estimated IC₅₀ value.

In some even more particular examples of testing the three concentrations, target activity may be followed using homogeneous, continuous (kinetic) assay formats that exploit the use of fluorophores to report on the functional competency status of the target. Where the target retains a catalytic function, a fluorescent product, either direct or surrogate, is formed, the fluorescence of which is directly proportional to the amount of product generated, enabling enzymatic activity to be followed in real time. Reactions are performed in a reaction buffer designed to provide a robust and stable environment enabling functional activity to be continuously monitored over time. Concentration response curves (CRCs; pseudo, targeted or standard) are independently generated directly in assay plates using the dispenser 306. All assay reagents (target, substrate/ligand, detection, etc.) are dispensed using the reagent dispenser 312. Plates are typically processed in the centrifuge 314 after each addition. Plates are immediately transferred to the plate reader 310 that is equipped with the sensor 308 to measure the required fluorescence wavelengths, and the change in fluorescence intensity monitored for over time.

Data analysis is performed autonomously using commercially-available and proprietary data processing software packages (e.g., R). The fluorescent signal resulting from either a single end point read or the steady-state change in fluorescence determined across the entire time course, is normalized to high (full activity) and low (full inhibition) controls prior to assessing assay robustness, using the industry-standard robust Z′ metric (plate level QC). Concentration response curves are fitted using a standard logistical four-parameter equation to determine IC₅₀. Pseudo-concentration response curves are fitted, locking the maximum asymptote, minimum asymptote and Hill slope at 100%, 0% and 1 respectively. 11-point targeted full curve fitting is assessed using the following constraint limits: maximum asymptote between 80 and 120%, minimum asymptote between -20 and 20% and Hill slope between 0.5 and 2.5. The process of fitting 11-point concentration response curve data involves (1) identification and exclusion of outliers where appropriate, (2) application of constraints for both maximum and/or minimum asymptotes and Hill slope as appropriate—unconstrained fit preferred, but where this is not possible, all combinations of constraint application are statistically compared and parameter values and errors for the most significant outcome are reported.

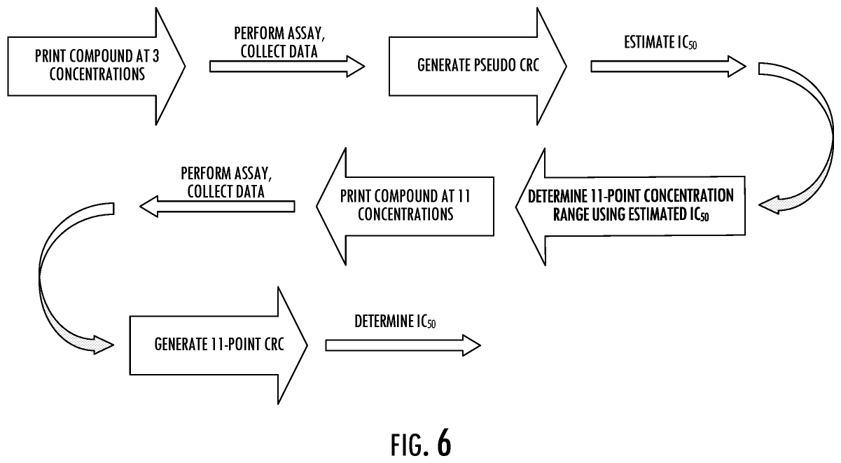

FIG. 6 is a flow diagram of some more specific example implementations of the present disclosure. Again, example implementations provide a pseudo-CRC technique to deliver a more precise IC₅₀ value for a given compound. This value provides information about a drug's efficacy which can help refine hit series in the early stages of drug development, ultimately leading to the generation of more potent and selective compounds with the potential to become novel medicines.

Step 1. As shown, a first step may include using the dispenser 306 to print a compound against a specific biological target into an assay plate in a contact free, non-serial manner. The D300e Digital Dispenser in particular is designed for picoliter to microliter, non-contact dispensing of liquids directly into an assay plate. Three distinct concentrations are printed for the compound with exquisite precision, such as at 40 μM, 1 μM and 25 nM. This 40-fold dilution may allow a wide range of potencies to be covered while reducing interference issues often observed at higher compound concentrations (>40 μM). It may also allow for equal ranges to be covered on either side of the 1 μM midpoint concentration.

After the compound has been printed and an assay thereby prepared, the assay may be performed with data including three response measurements collected using the sensor 308. A pseudo CRC may then be constructed from the three concentrations and the three response measurements.

Step 2. In a second step, the pseudo CRC from the first step may be analyzed using a commercially-available data analysis software package such as GraphPad Prism or Grafit to visualize the pseudo CRC with one point per respective concentration tested with concentration of compound (molar) on the x axis and response measurement—e.g., robust inhibition (%)—on the y axis. The pseudo CRC may be fitted using the Hill equation (represented below) in which a regression model of four parameters can be used to obtain the IC₅₀ value. Unlike standard four parameter analysis where all parameters are left unconstrained (also referred to as floated), some examples may only allow the IC₅₀ parameter to float with the other three parameters constrained.

In particular, the Hill equation may be represented as follows:

$$γ = \frac{Range}{1 + {( \frac{x}{{IC}_{50}} )}^{s}} + Background$$

In the Hill equation, Background is a fully inhibited rate where the biological target of interest is fully inhibited, Range is the difference between the uninhibited rate (biological target of interest is uninhibited) and the Background, and s represents the Hill slope (steepness of a concentration response curve). The fitting constraints applied to achieve an estimate IC₅₀ for the compound tested may include:

Maximum asymptote locked at a value of 100

Minimum asymptote locked at a value of 0

Hill slope set to 1, which assumes 1:1 chemical stoichiometry The IC₅₀ variable may be left to ‘float’ freely so that it may be estimated.

Step 3. The estimated IC₅₀ value determined in the second step may be used in a third step to determine an individualized concentration range with a plurality of concentrations, such as eleven concentrations, compared to the three concentrations from the first step. A derivation of eleven concentrations for a 1-9-1 spacing according to some examples is provided below. The eleven or other plurality of concentrations may be tested in a manner similar to the earlier, three concentrations. Testing the eleven concentration points will ultimately allow a freely fitted IC₅₀ value to be calculated which could help make informed decisions about which specific hit series (for the process over a plurality of compounds) should be progressed down the drug discovery pipeline, highlighting the broader applications of this method.

The eleven concentrations may be tested to enable a full curve assessment in which a better-defined CRC may be obtained. The CRC, then, may provide highly-precise information about compound potency, Hill slope and behavior. For the process performed over a plurality of compounds, the information provided by the CRC may highlight those of the plurality of compounds that have the potential to reach candidate selection, the phase of drug discovery which marks the start of the clinical trials phase for experimental drugs. With a 1-9-1 spacing of data points, concentrations 2 to 10 of the eleven-point range may cover the aforementioned 13.65 to 86.35% robust inhibition and span the Hill slope, the portion of the curve that provides the most valuable data.

Once the eleven concentration points have been determined, the compound may be reprinted at these respective concentrations using the dispenser 306 to prepare an assay plate. As in the first step, the assay may then be performed, with data including response measurements collected using the sensor 308. The data may be analyzed in a manner similar to before to produce an eleven-point CRC, with concentration of compound on the x axis (Molar) and response measurement—e.g., robust inhibition (%)—on the y axis.

In some examples, a few assumptions may be made in this step, namely, unless otherwise stated, the compound or compounds being tested are assumed to bind competitively with respect to their biological targets. This means that the compound has an affinity for the substrate binding site of the biological target of interest, competing directly with the target's physiological substrate. Another assumption may be that because molecular events are occurring in solution, the rate of compound binding to biological target may be assumed to be no more than an order of magnitude slower than the limit of diffusion, equivalent to 10⁸M⁻¹.s⁻¹.

Derivation of an eleven-point concentration range (for 1-9-1 spacing). In some examples, for an eleven-point concentration range, the uppermost concentration (point 1) for the compound (notationally [I]ₘₐₓ(M)) may be the concentration achieved by printing the maximum volume of compound the dispenser 306 is designed to dispense. This in turn may be limited the % DMSO used in the assay as well as the assay volume. To clarify this, for an assay with a 1% DMSO limit, the maximum test concentration possible may be 100 μM from a 10 mM stock solution of compound. Using higher concentration stock solutions of compounds within an assay allows access to data at increased concentrations which is likely to be more relevant for weaker compounds.

The lowermost concentration of the eleven-point range (point 11) (notationally [I]ₘᵢₙ(M)) may depend on the minimum concentration that the dispenser 306 is designed to dispense. This may correspond to the minimum concentration the dispenser in use can print precisely (specifically, a single droplet of compound) and the stock concentration of compound. This may also be the lowest concentration that can equilibrate within the assay duration based on the assumed competitive binding kinetics of a given compound.

In some examples, generation of a targeted, eleven-point CRC may include calculation of a concentration of a stock solution of the compound. In this regard, the stock solution of the compound may be a concentrated solution that will be diluted to form less concentrated solutions for assay use. In some examples, it may be assumed that 10 mM will be the maximal achievable concentration of stock compound, factoring compound solubility that decreases above this concentration. A recommended stock concentration may be calculated by multiplying the compound's estimated IC₅₀ (calculated in step 2) by 10,000; and this value may be kept at or below 10 mM.

To understand how concentration points 2-10 may be determined, a number of variables may first be defined. These variables may include one or more of a targeted titration factor, an upper [I] limit (M), a lower [I] limit (M), an upper IC₅₀ limit (M), or a lower IC₅₀ limit (M). The targeted titration factor is the fold dilution to achieve a particular range across the nine concentration points from 2 to 10. This range may be a 40-fold range across the nine concentration points; and given a 40-fold dilution factor, the targeted titration factor may equal 1.5858 (1.5858⁸ =40).

If the estimated IC₅₀ value multiplied by the square root of 40 (method's dilution factor) is greater than the [I]ₘₐₓ/targeted titration factor, the upper [I] limit may be determined to equal [I]ₘₐₓ/targeted titration factor; otherwise, the upper [I] limit may be set equal to [I]ₘₐₓ. Similarly, if the estimated IC₅₀ divided by the square root of 40 is less than [I]ₘᵢₙ×targeted titration factor, the lower [I] limit may be determined to equal [I]ₘᵢₙ×targeted titration factor; and otherwise, the lower [I] limit may be set to equal [I]ₘᵢₙ. The upper IC₅₀ limit (M) may be set equal to [I]ₘₐₓ divided by the targeted titration factor squared; and the lower IC₅₀ limit (M) may be set equal to [I]ₘᵢₙ multiplied by the targeted titration factor squared.

Given the above variables, concentration points 1 and 11 (uppermost and lowermost concentrations of the eleven-point concentration range, respectively) may be set to respectively _{[I]max} (point 1) and [I]ₘᵢₙ (point 11). The midpoint one of the eleven concentrations (point 6) may be determined based on the estimated IC₅₀ value. In this regard, the midpoint concentration may be set equal to the estimated IC₅₀ value when the estimated IC₅₀ value is between the uppermost and lowermost concentrations; and otherwise, the midpoint concentration may be set equal to a value that is equidistant between the upper IC₅₀ limit (M) and the lower IC₅₀ limit (M).

Those of the eleven concentrations above the midpoint (points 2, 3, 4 and 5 may be determined as follows. Point 2 may be determined to equal the midpoint concentration (point 6) x targeted titration factor to the power of 4; point 3 may be determined to equal the midpoint concentration x targeted titration factor to the power of 3; point 4 may be determined to equal the midpoint concentration x targeted titration factor to the power of 2; and point 5 may be determined to equal the midpoint concentration×targeted titration factor.

Some of those of the eleven concentrations below the midpoint (points 7, 8 and 9 may be determined as follows. Point 7 may be determined to equal the midpoint concentration (point 6)/targeted titration factor; point 8 may be determined to equal the midpoint concentration/targeted titration factor to the power of 2; and point 9 may be determined to equal the midpoint concentration/targeted titration factor to the power of 3. The remaining concentration point below the midpoint (point 10) may be determined in a similar manner, but with further consideration of the estimated IC₅₀ value. That is, if the estimated IC₅₀ value is less than the upper [I] limit, point 10 may be determined to equal the midpoint concentration (point 6)/targeted titration factor to the power of 4; otherwise, point 10 may be set to a value equally spaced between point 9 and the lowermost concentration (point 11).

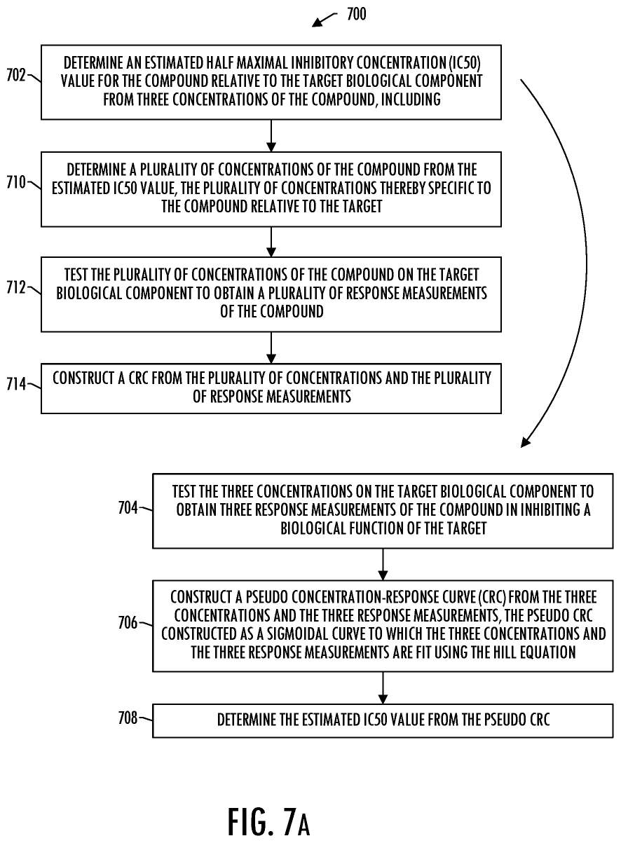

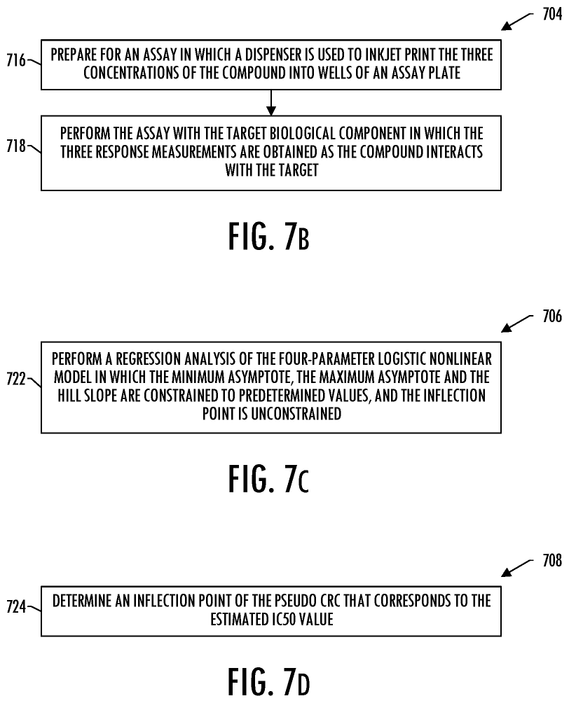

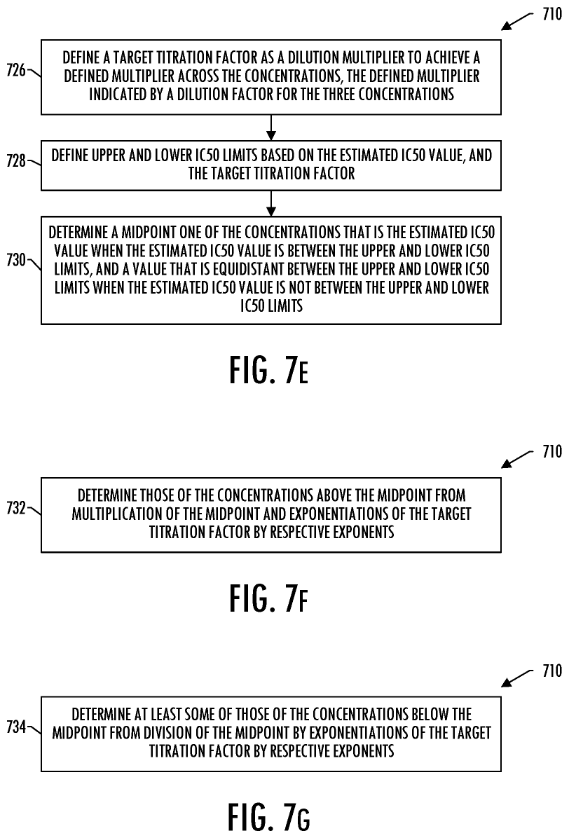

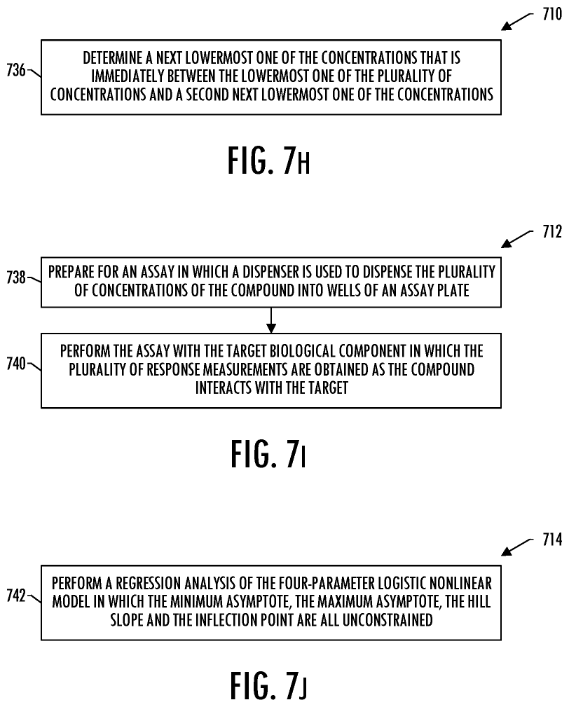

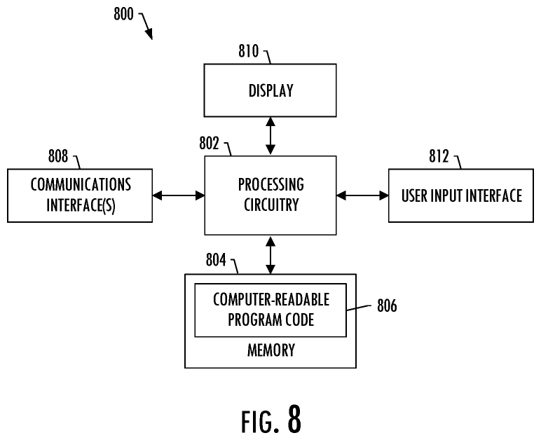

FIGS. 7A-7J are flowcharts illustrating various steps in a method 700 of screening a compound relative to a target biological component, according to various example implementations of the present disclosure. The method includes determining an estimated half maximal inhibitory concentration (IC₅₀) value for the compound relative to the target biological component from three concentrations of the compound, as shown at block 702 of FIG. 7A. This includes testing the three concentrations on the target biological component to obtain three response measurements of the compound in inhibiting a biological function of the target, as shown at block 704. A pseudo concentration-response curve (CRC) is constructed from the three concentrations and the three response measurements, and as a sigmoidal curve to which the three concentrations and the three response measurements are fit using the Hill equation, as shown at block 706. And the the estimated IC₅₀ value is determined from the pseudo CRC, as shown at block 708.

As also shown, the method 700 includes determining a plurality of concentrations of the compound from the estimated IC₅₀ value, the plurality of concentrations thereby specific to the compound relative to the target, as shown at block 710. The method includes testing the plurality of concentrations of the compound on the target biological component to obtain a plurality of response measurements of the compound, as shown at block 712. And the method includes constructing a CRC from the plurality of concentrations and the plurality of response measurements, as shown at block 714.

In some examples, the three concentrations have a dilution factor that is a defined multiplier for the three concentrations, and the three concentrations include a lowermost concentration, an intermediate concentration that is a multiple of the lowermost concentration for the defined multiplier, and an uppermost concentration that is a multiple of the intermediate concentration for the defined multiplier.

In some examples, the dilution factor is 10, the lowermost concentration is 100 nanomolar, the intermediate concentration is 1 micromolar, and the uppermost concentration is 10 micromolar.

In some examples, the dilution factor is 40, the lowermost concentration is 25 nanomolar, the intermediate concentration is 1 micromolar, and the uppermost concentration is 40 micromolar.

In some examples, the three concentrations are selected to cover a range of inhibition measurements from 13.65% to 86.25% when the plurality of concentrations are tested, and span a Hill slope of the CRC when constructed from the plurality of concentrations and the plurality of response measurements.

In some examples, testing the three concentrations at block 704 includes preparing for an assay in which a dispenser is used to dispense the three concentrations of the compound into wells of an assay plate, as shown at block 716 of FIG. 7B. In some of these examples, the assay is then performed with the target biological component in which the three response measurements are obtained as the compound interacts with the target, as shown at block 718.

In some examples, the Hill equation is a four-parameter logistic nonlinear model with parameters of the sigmoidal curve including a minimum asymptote, a maximum asymptote, an inflection point that corresponds to the IC₅₀, and a Hill slope. In some of these examples, constructing the pseudo CRC at block 706 includes performing a regression analysis of the four-parameter logistic nonlinear model in which the minimum asymptote, the maximum asymptote and the Hill slope are constrained to predetermined values, and the inflection point is unconstrained, as shown at block 722 of FIG. 7C.

In some examples, determining the estimated IC₅₀ value at block 708 includes determining an inflection point of the pseudo CRC that corresponds to the estimated IC₅₀ value, as shown at block 724 of FIG. 7D.

In some examples, the plurality of concentrations of the compound are tested at block 712 using a dispenser to dispense the plurality of concentrations, and uppermost and lowermost ones of the plurality of concentrations are determined based on maximum and minimum concentrations the dispenser is designed to dispense. And in some further examples, the dispenser is a digital dispenser, acoustic dispenser, piezo dispenser, or positive displacement dispenser.

In some examples, the plurality of concentrations includes concentrations that are between uppermost and lowermost ones of the plurality of concentrations. In some of these examples, determining the plurality of concentrations at block 710 includes defining a target titration factor as a dilution multiplier to achieve a defined multiplier across the concentrations, the defined multiplier indicated by a dilution factor for the three concentrations, as shown at block 726 of FIG. 7E. Upper and lower IC₅₀ limits are defined based on the estimated IC₅₀ value, and the target titration factor, as shown at block 728. And the method includes determining a midpoint one of the concentrations that is the estimated IC₅₀ value when the estimated IC₅₀ value is between the upper and lower IC₅₀ limits, and a value that is equidistant between the upper and lower IC₅₀ limits when the estimated IC₅₀ value is not between the upper and lower IC₅₀ limits, as shown at block 730.

In some examples, determining the plurality of concentrations further at block 710 includes determining those of the concentrations above the midpoint from multiplication of the midpoint and exponentiations of the target titration factor by respective exponents, as shown at block 732 of FIG. 7F.

In some examples, determining the plurality of concentrations further at block 710 includes determining at least some of those of the concentrations below the midpoint from division of the midpoint by exponentiations of the target titration factor by respective exponents, as shown at block 734 of FIG. 7G.

In some examples, determining the plurality of concentrations further at block 710 includes determining a next lowermost one of the concentrations that is immediately between the lowermost one of the plurality of concentrations and a second next lowermost one of the concentrations, as shown at block 736 of FIG. 7H. In some of these examples, the next lowermost one of the concentrations is determined from division of the midpoint by an exponentiation of the target titration factor when the estimated IC₅₀ value is less than an upper concentration limit, and as the value that is equidistant between the lowermost and the second next lowermost ones of the plurality of concentrations when the estimated IC₅₀ value is greater than an upper concentration limit.

In some examples, testing the plurality of concentrations at block 712 includes preparing for an assay in which a dispenser is used to dispense the plurality of concentrations of the compound into wells of an assay plate, as shown at block 738 of FIG. 71. And the assay is performed with the target biological component in which the plurality of response measurements are obtained as the compound interacts with the target, as shown at block 740.

In some examples, the assay is prepared at block 738 in which the dispenser is a digital dispenser used to print and thereby dispense the plurality of concentrations of the compound.

In some examples, the CRC is constructed at block 714 as a second sigmoidal curve to which the plurality of concentrations and the plurality of response measurements are fit using the Hill equation.

In some examples, the Hill equation is a four-parameter logistic nonlinear model with parameters of the sigmoidal curve including a minimum asymptote, a maximum asymptote, an inflection point that corresponds to the IC₅₀, and a Hill slope. And in some of these examples, constructing the CRC at block 714 includes performing a regression analysis of the four-parameter logistic nonlinear model in which the minimum asymptote, the maximum asymptote, the Hill slope and the inflection point are all unconstrained, as shown at block 742 of FIG. 7J.

In some examples, the three concentrations are tested at block 704 in a laboratory with laboratory equipment including a dispenser controlled to dispense the three concentrations into wells of a first assay plate, and at least one sensor from which the three response measurements are obtained. And in some of these examples, the plurality of concentrations are tested at block 712 in the laboratory with the laboratory equipment in which the dispenser is controlled to dispense the plurality of concentrations into the wells of a second assay plate, and the plurality of response measurements are obtained from the at least one sensor.

According to example implementations of the present disclosure, the system 300 and its subsystems including the computer 302 and laboratory equipment 304 may be implemented by various means. Means for implementing the system and its subsystems may include hardware, alone or under direction of one or more computer programs from a computer-readable storage medium. In some examples, one or more apparatuses may be configured to function as or otherwise implement the system and its subsystems shown and described herein. In examples involving more than one apparatus, the respective apparatuses may be connected to or otherwise in communication with one another in a number of different manners, such as directly or indirectly via a wired or wireless network or the like.

FIG. 8 illustrates an apparatus 800 according to some example implementations of the present disclosure. Generally, an apparatus of exemplary implementations of the present disclosure may comprise, include or be embodied in one or more fixed or portable electronic devices. Examples of suitable electronic devices include a smartphone, tablet computer, laptop computer, desktop computer, workstation computer, server computer or the like. The apparatus may include one or more of each of a number of components such as, for example, processing circuitry 802 (e.g., processor unit) connected to a memory 804 (e.g., storage device).

The processing circuitry 802 may be composed of one or more processors alone or in combination with one or more memories. The processing circuitry is generally any piece of computer hardware that is capable of processing information such as, for example, data, computer programs and/or other suitable electronic information. The processing circuitry is composed of a collection of electronic circuits some of which may be packaged as an integrated circuit or multiple interconnected integrated circuits (an integrated circuit at times more commonly referred to as a “chip”). The processing circuitry may be configured to execute computer programs, which may be stored onboard the processing circuitry or otherwise stored in the memory 804 (of the same or another apparatus).

The processing circuitry 802 may be a number of processors, a multi-core processor or some other type of processor, depending on the particular implementation. Further, the processing circuitry may be implemented using a number of heterogeneous processor systems in which a main processor is present with one or more secondary processors on a single chip. As another illustrative example, the processing circuitry may be a symmetric multi-processor system containing multiple processors of the same type. In yet another example, the processing circuitry may be embodied as or otherwise include one or more ASICs, FPGAs or the like. Thus, although the processing circuitry may be capable of executing a computer program to perform one or more functions, the processing circuitry of various examples may be capable of performing one or more functions without the aid of a computer program. In either instance, the processing circuitry may be appropriately programmed to perform functions or operations according to example implementations of the present disclosure.

The memory 804 is generally any piece of computer hardware that is capable of storing information such as, for example, data, computer programs (e.g., computer-readable program code 806) and/or other suitable information either on a temporary basis and/or a permanent basis. The memory may include volatile and/or non-volatile memory, and may be fixed or removable. Examples of suitable memory include random access memory (RAM), read-only memory (ROM), a hard drive, a flash memory, a thumb drive, a removable computer diskette, an optical disk, a magnetic tape or some combination of the above. Optical disks may include compact disk—read only memory (CD-ROM), compact disk—read/write (CD-R/W), DVD or the like. In various instances, the memory may be referred to as a computer-readable storage medium. The computer-readable storage medium is a non-transitory device capable of storing information, and is distinguishable from computer-readable transmission media such as electronic transitory signals capable of carrying information from one location to another. Computer-readable medium as described herein may generally refer to a computer-readable storage medium or computer-readable transmission medium.

In addition to the memory 804, the processing circuitry 802 may also be connected to one or more interfaces for displaying, transmitting and/or receiving information. The interfaces may include a communications interface 808 (e.g., communications unit) and/or one or more user interfaces. The communications interface may be configured to transmit and/or receive information, such as to and/or from other apparatus(es), network(s) or the like. The communications interface may be configured to transmit and/or receive information by physical (wired) and/or wireless communications links. Examples of suitable communication interfaces include a network interface controller (NIC), wireless NIC (WNIC) or the like.

The user interfaces may include a display 810 and/or one or more user input interfaces 812 (e.g., input/output unit). The display may be configured to present or otherwise display information to a user, suitable examples of which include a liquid crystal display (LCD), light-emitting diode display (LED), plasma display panel (PDP) or the like. The user input interfaces may be wired or wireless, and may be configured to receive information from a user into the apparatus, such as for processing, storage and/or display. Suitable examples of user input interfaces include a microphone, image or video capture device, keyboard or keypad, joystick, touch-sensitive surface (separate from or integrated into a touchscreen), biometric sensor or the like. The user interfaces may further include one or more interfaces for communicating with peripherals such as printers, scanners or the like.

As indicated above, program code instructions may be stored in memory, and executed by processing circuitry that is thereby programmed, to implement functions of the systems, subsystems, tools and their respective elements described herein. As will be appreciated, any suitable program code instructions may be loaded onto a computer or other programmable apparatus from a computer-readable storage medium to produce a particular machine, such that the particular machine becomes a means for implementing the functions specified herein. These program code instructions may also be stored in a computer-readable storage medium that can direct a computer, a processing circuitry or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions may be retrieved from a computer-readable storage medium and loaded into a computer, processing circuitry or other programmable apparatus to configure the computer, processing circuitry or other programmable apparatus to execute operations to be performed on or by the computer, processing circuitry or other programmable apparatus.

Retrieval, loading and execution of the program code instructions may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions may produce a computer-implemented process such that the instructions executed by the computer, processing circuitry or other programmable apparatus provide operations for implementing functions described herein.

Execution of instructions by a processing circuitry, or storage of instructions in a computer-readable storage medium, supports combinations of operations for performing the specified functions. In this manner, an apparatus 800 may include a processing circuitry 802 and a computer-readable storage medium or memory 804 coupled to the processing circuitry, where the processing circuitry is configured to execute computer-readable program code 806 stored in the memory. It will also be understood that one or more functions, and combinations of functions, may be implemented by special purpose hardware-based computer systems and/or processing circuitry which perform the specified functions, or combinations of special purpose hardware and program code instructions.

As explained above and reiterated below, the present disclosure includes, without limitation, the following example implementations.

Clause 1. A system for screening a compound relative to a target biological component, the system comprising a computer including: a memory configured to store computer-readable program code; and processing circuitry configured to access the memory, and execute the computer-readable program code to cause the computer to at least: determine an estimated half maximal inhibitory concentration (IC₅₀) value for the compound relative to the target biological component from three concentrations of the compound, including the computer caused to: test the three concentrations on the target biological component to obtain three response measurements of the compound in inhibiting a biological function of the target; construct a pseudo concentration-response curve (CRC) from the three concentrations and the three response measurements, the pseudo CRC constructed as a sigmoidal curve to which the three concentrations and the three response measurements are fit using the Hill equation; and determine the estimated IC₅₀ value from the pseudo CRC; determine a plurality of concentrations of the compound from the estimated IC₅₀ value, the plurality of concentrations thereby specific to the compound relative to the target; test the plurality of concentrations of the compound on the target biological component to obtain a plurality of response measurements of the compound; and construct a CRC from the plurality of concentrations and the plurality of response measurements.

Clause 2. The system of clause 1, wherein the three concentrations have a dilution factor that is a defined multiplier for the three concentrations, and the three concentrations include a lowermost concentration, an intermediate concentration that is a multiple of the lowermost concentration for the defined multiplier, and an uppermost concentration that is a multiple of the intermediate concentration for the defined multiplier.

Clause 3. The system of clause 2, wherein the dilution factor is 10, the lowermost concentration is 100 nanomolar, the intermediate concentration is 1 micromolar, and the uppermost concentration is 10 micromolar.

Clause 4. The system of clause 2 or clause 3, wherein the dilution factor is 40, the lowermost concentration is 25 nanomolar, the intermediate concentration is 1 micromolar, and the uppermost concentration is 40 micromolar.

Clause 5. The system of any of clauses 1 to 4, wherein the three concentrations are selected to cover a range of inhibition measurements from 13.65% to Clause 86.25% when the plurality of concentrations are tested, and span a Hill slope of the CRC when constructed from the plurality of concentrations and the plurality of response measurements.

Clause 6. The system of any of clauses 1 to 5, wherein the system further comprises a dispenser, and the computer caused to test the three concentrations includes the computer caused to: prepare for an assay in which the dispenser is used to dispense the three concentrations of the compound into wells of an assay plate; and perform the assay with the target biological component in which the three response measurements are obtained as the compound interacts with the target.

Clause 7: The system of any of clauses 1 to 6, wherein the Hill equation is a four-parameter logistic nonlinear model with parameters of the sigmoidal curve including a minimum asymptote, a maximum asymptote, an inflection point that corresponds to the IC₅₀, and a Hill slope, and wherein the computer caused to construct the pseudo CRC includes the computer caused to perform a regression analysis of the four-parameter logistic nonlinear model in which the minimum asymptote, the maximum asymptote and the Hill slope are constrained to predetermined values, and the inflection point is unconstrained.

Clause 8. The system of any of clauses Ito 7, wherein the computer caused to determine the estimated IC₅₀ value includes the computer caused to determine an inflection point of the pseudo CRC that corresponds to the estimated IC₅₀ value.

Clause 9. The system of any of clauses 1 to 8, wherein the system further comprises a dispenser, and the plurality of concentrations of the compound are tested using the dispenser to dispense the plurality of concentrations, and uppermost and lowermost ones of the plurality of concentrations are determined based on maximum and minimum concentrations the dispenser is designed to dispense.

Clause 10. The system of clause 9, wherein the dispenser is a digital dispenser, acoustic dispenser, piezo dispenser, or positive displacement dispenser.

Clause 11. The system of clause 9 or clause 10, wherein the plurality of concentrations includes concentrations that are between uppermost and lowermost ones of the plurality of concentrations, and the computer caused to determine the plurality of concentrations includes the computer caused to: define a target titration factor as a dilution multiplier to achieve a defined multiplier across the concentrations, the defined multiplier indicated by a dilution factor for the three concentrations; define upper and lower IC₅₀ limits based on the estimated IC₅₀ value, and the target titration factor; and determine a midpoint one of the concentrations that is the estimated IC₅₀ value when the estimated IC₅₀ value is between the upper and lower IC₅₀ limits, and a value that is equidistant between the upper and lower IC₅₀ limits when the estimated IC₅₀ value is not between the upper and lower IC₅₀ limits.

Clause 12. The system of clause 11, wherein the computer caused to determine the plurality of concentrations further includes the computer caused to determine those of the concentrations above the midpoint from multiplication of the midpoint and exponentiations of the target titration factor by respective exponents.

Clause 13. The system of clause 11 or clause 12, wherein the computer caused to determine the plurality of concentrations further includes the computer caused to determine at least some of those of the concentrations below the midpoint from division of the midpoint by exponentiations of the target titration factor by respective exponents.

Clause 14. The system of any of clauses 11 to 13, wherein the computer caused to determine the plurality of concentrations further includes the computer caused to determine a next lowermost one of the concentrations that is immediately between the lowermost one of the plurality of concentrations and a second next lowermost one of the concentrations, and wherein the next lowermost one of the concentrations is determined from division of the midpoint by an exponentiation of the target titration factor when the estimated IC₅₀ value is less than an upper concentration limit, and as the value that is equidistant between the lowermost and the second next lowermost ones of the plurality of concentrations when the estimated IC₅₀ value is greater than an upper concentration limit.

Clause 15. The system of any of clauses 1 to 14, wherein the system further comprises a dispenser, and the computer caused to test the plurality of concentrations includes the computer caused to: prepare for an assay in which the dispenser is used to dispense the plurality of concentrations of the compound into wells of an assay plate; and perform the assay with the target biological component in which the plurality of response measurements are obtained as the compound interacts with the target.

Clause 16. The system of clause 15, wherein the assay is prepared in which the dispenser is a digital dispenser used to print and thereby dispense the plurality of concentrations of the compound.

Clause 17. The system of any of clauses 1 to 16, wherein the CRC is constructed as a second sigmoidal curve to which the plurality of concentrations and the plurality of response measurements are fit using the Hill equation.

Clause 18. The system of clause 17, wherein the Hill equation is a four-parameter logistic nonlinear model with parameters of the sigmoidal curve including a minimum asymptote, a maximum asymptote, an inflection point that corresponds to the IC₅₀, and a Hill slope, and wherein the apparatus caused to construct the CRC includes the apparatus caused to perform a regression analysis of the four-parameter logistic nonlinear model in which the minimum asymptote, the maximum asymptote, the Hill slope and the inflection point are all unconstrained.

Clause 19. The system of any of clauses 1 to 18, wherein the system further comprises laboratory equipment including a. dispenser and at least one sensor, and the three concentrations are tested in a laboratory in which the dispenser is controllable to dispense the three concentrations into wells of a first assay plate, and the at least one sensor is configured to obtain the three response measurements are obtained, and wherein the plurality of concentrations are tested in the laboratory with the laboratory equipment in which the dispenser is controllable to dispense the plurality of concentrations into the wells of a second assay plate, and the plurality of response measurements are obtained from the at least one sensor.

Clause 20. A method of screening a compound relative to a target biological component, the method comprising: determining an estimated half maximal inhibitory concentration (IC₅₀) value for the compound relative to the target biological component from three concentrations of the compound, including: testing the three concentrations on the target biological component to obtain three response measurements of the compound in inhibiting a biological function of the target; constructing a pseudo concentration-response curve (CRC) from the three concentrations and the three response measurements, the pseudo CRC constructed as a sigmoidal curve to which the three concentrations and the three response measurements are fit using the Hill equation; and determining the estimated 1C₅₀ value from the pseudo CRC; determining a plurality of concentrations of the compound from the estimated IC₅₀ value, the plurality of concentrations thereby specific to the compound relative to the target; testing the plurality of concentrations of the compound on the target biological component to obtain a plurality of response measurements of the compound; and constructing a CRC from the plurality of concentrations and the plurality of response measurements.

Clause 21. The method of clause 20, wherein the three concentrations have a dilution factor that is a defined multiplier for the three concentrations, and the three concentrations include a lowermost concentration, an intermediate concentration that is a multiple of the lowermost concentration for the defined multiplier, and an uppermost concentration that is a multiple of the intermediate concentration for the defined multiplier.

Clause 22. The method of clause 21, wherein the dilution factor is 10, the lowermost concentration is 100 nanomolar, the intermediate concentration is I micromolar, and the uppermost concentration is 10 micromolar.

Clause 23. The method of clause 21 or clause 22, wherein the dilution factor is 40, the lowermost concentration is 25 nanomolar, the intermediate concentration is 1 micromolar, and the uppermost concentration is 40 micromolar,

Clause 24. The method of any of clauses 20 to 23, wherein the three concentrations are selected to cover a range of inhibition measurements from 13.65% to Clause 86.25% when the plurality of concentrations are tested, and span a Hill slope of the CRC when constructed from the plurality of concentrations and the plurality of response measurements.

Clause 25. The method of any of clauses 20 to 24, wherein testing the three concentrations includes: preparing for an assay in which a dispenser is used to dispense the three concentrations of the compound into wells of an assay plate; and performing the assay with the target biological component in which the three response measurements are obtained as the compound interacts with the target.

Clause 26. The method of any of clauses 20 to 25, wherein the Hill equation is a four-parameter logistic nonlinear model with parameters of the sigmoidal curve including a minimum asymptote, a maximum asymptote, an inflection point that corresponds to the IC₅₀, and a Hill slope, and wherein constructing the pseud© CRC includes performing a regression analysis of the four-parameter logistic nonlinear model in which the minimum asymptote, the maximum asymptote and the slope are constrained to predetermined values, and the inflection point is unconstrained.

Clause 27. The method of any of clauses 20 to 26, wherein determining the estimated IC₅₀ value includes determining an inflection point of the pseudo CRC that corresponds to the estimated IC₅₀ value.

Clause 28. The method of any of clauses 20 to 27, wherein the plurality of concentrations of the compound are tested using a dispenser to dispense the plurality of concentrations, and uppermost and lowermost ones of the plurality of concentrations are determined based on maximum and minimum concentrations the dispenser is designed to dispense.

Clause 29. The method of clause 28, wherein the dispenser is a digital dispenser, acoustic dispenser, piezo dispenser, or positive displacement dispenser.

Clause 30. The method of clause 28 or clause 29_{;} wherein the plurality of concentrations includes concentrations that are between uppermost and lowermost ones of the plurality of concentrations, and determining the plurality of concentrations includes: defining a target titration factor as a dilution multiplier to achieve a defined multiplier across the concentrations, the defined multiplier indicated by a dilution factor for the three concentrations; defining upper and lower IC₅₀ limits based on the estimated IC₅₀ value, and the target titration factor; and determining a. midpoint one of the concentrations that is the estimated IC₅₀ value when the estimated IC₅₀ value is between the upper and lower IC₅₀ limits, and a. value that is equidistant between the upper and lower IC₅₀ limits when the estimated IC₅₀ value is not between the upper and lower IC₅₀ limits.

Clause 31. The method of clause 30, wherein determining the plurality of concentrations further includes determining those of the concentrations above the midpoint from multiplication of the midpoint and exponentiations of the target titration factor by respective exponents.

Clause 32. The method of clause 30 or clause 31, wherein determining the plurality of concentrations further includes determining at least some of those of the concentrations below the midpoint from division of the midpoint by exponentiations of the target titration factor by respective exponents.

Clause 33. The method of any of clauses 30 to 32, wherein determining the plurality of concentrations further includes determining a next lowermost one of the concentrations that is immediately between the lowermost one of the plurality of concentrations and a second next lowermost one of the concentrations, and wherein the next lowermost one of the concentrations is determined from division of the midpoint by an exponentiation of the target titration factor when the estimated 10₅₀ value is less than an upper concentration limit, and as the value that is equidistant between the lowermost and the second next lowermost ones of the plurality of concentrations when the estimated IC₅₀ value is greater than an upper concentration limit.

Clause 34. The method of any of clauses 20 to 33, wherein testing the plurality of concentrations includes: preparing for an assay in which a dispenser is used to dispense the plurality of concentrations of the compound into wells of an assay plate; and performing the assay with the target biological component in which the plurality of response measurements are obtained as the compound interacts with the target.

Clause 35. The method of clause 34, wherein the assay is prepared in which the dispenser is a digital dispenser used to print and thereby dispense the plurality of concentrations of the compound.

Clause 36. The method of any of clauses 20 to 35, wherein the CRC is constructed as a second sigmoidal curve to which the plurality of concentrations and the plurality of response measurements are fit using the Hill equation.

Clause 37. The method of clause 36, wherein the Hill equation is a four-parameter logistic nonlinear model with parameters of the sigmoidal curve including a minimum asymptote, a maximum asymptote, an inflection point that corresponds to the IC₅₀, and a Hi IL slope, and wherein constructing the CRC includes performing a regression analysis of the four-parameter logistic nonlinear model in which the minimum asymptote, the maximum asymptote, the Hill slope and the inflection point are all unconstrained.

Clause 38. The method of any of clauses 20 to 37, wherein the three concentrations are tested in a laboratory with laboratory equipment including a dispenser controlled to dispense the three concentrations into wells of a first assay plate, and at least one sensor from which the three response measurements are obtained, and wherein the plurality of concentrations are tested in the laboratory with the laboratory equipment in which the dispenser is controlled to dispense the plurality of concentrations into the wells of a second assay plate, and the plurality of response measurements are obtained from the at least one sensor.

Many modifications and other implementations of the disclosure set forth herein will come to mind to one skilled in the art to which the disclosure pertains having the benefit of the teachings presented in the foregoing description and the associated figures. Therefore, it is to be understood that the disclosure is not to be limited to the specific implementations disclosed and that modifications and other implementations are intended to be included within the scope of the appended claims. Moreover, although the foregoing description and the associated figures describe example implementations in the context of certain example combinations of elements and/or functions, it should be appreciated that different combinations of elements and/or functions may be provided by alternative implementations without departing from the scope of the appended claims. in this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A system for screening a compound relative to a target biological component, the system comprising a computer including:
a memory configured to store computer-readable program code; and
processing circuitry configured to access the memory, and execute the computer-readable program code to cause the computer to at least:
determine an estimated half maximal inhibitory concentration (IC₅₀) value for the compound relative to the target biological component from three concentrations of the compound, including the computer caused to:
test the three concentrations on the target biological component to obtain three response measurements of the compound in inhibiting a biological function of the target;
construct a pseudo concentration-response curve (CRC) from the three concentrations and the three response measurements, the pseudo CRC constructed as a sigmoidal curve to which the three concentrations and the three response measurements are fit using the Hill equation; and
determine the estimated IC₅₀ value from the pseudo CRC;
determine a plurality of concentrations of the compound from the estimated IC₅₀ value, the plurality of concentrations thereby specific to the compound relative to the target;
test the plurality of concentrations of the compound on the target biological component to obtain a plurality of response measurements of the compound; and
construct a CRC from the plurality of concentrations and the plurality of response measurements.

2. The system of claim 1, wherein the three concentrations have a dilution factor that is a defined multiplier for the three concentrations, and the three concentrations include a lowermost concentration, an intermediate concentration that is a multiple of the lowermost concentration for the defined multiplier, and an uppermost concentration that is a multiple of the intermediate concentration for the defined multiplier.

3. The system of claim 1, wherein the Hill equation is a four-parameter logistic nonlinear model with parameters of the sigmoidal curve including a minimum asymptote, a maximum asymptote, an inflection point that corresponds to the IC₅₀, and a Hill slope, and
wherein the computer caused to construct the pseudo CRC includes the computer caused to perform a regression analysis of the four-parameter logistic nonlinear model in which the minimum asymptote, the maximum asymptote and the Hill slope are constrained to predetermined values, and the inflection point is unconstrained.

4. The system of claim 1, wherein the computer caused to determine the estimated IC₅₀ value includes the computer caused to determine an inflection point of the pseudo CRC that corresponds to the estimated IC₅₀ value.

5. The system of claim 1, wherein the system further comprises a dispenser, and the plurality of concentrations of the compound are tested using the dispenser to dispense the plurality of concentrations, and uppermost and lowermost ones of the plurality of concentrations are determined based on maximum and minimum concentrations the dispenser is designed to dispense.

6. The system of claim 5, wherein the plurality of concentrations includes concentrations that are between uppermost and lowermost ones of the plurality of concentrations, and the computer caused to determine the plurality of concentrations includes the computer caused to:
define a target titration factor as a dilution multiplier to achieve a defined multiplier across the concentrations, the defined multiplier indicated by a dilution factor for the three concentrations;
define upper and lower IC₅₀ limits based on the estimated IC₅₀ value, and the target titration factor; and
determine a midpoint one of the concentrations that is the estimated IC₅₀ value when the estimated IC₅₀ value is between the upper and lower IC₅₀ limits, and a value that is equidistant between the upper and lower IC₅₀ limits when the estimated IC₅₀ value is not between the upper and lower IC₅₀ limits.

7. The system of claim 6, wherein the computer caused to determine the plurality of concentrations further includes the computer caused to determine those of the concentrations above the midpoint from multiplication of the midpoint and exponentiations of the target titration factor by respective exponents.

8. The system of claim 6, wherein the computer caused to determine the plurality of concentrations further includes the computer caused to determine at least some of those of the concentrations below the midpoint from division of the midpoint by exponentiations of the target titration factor by respective exponents.

9. The system of claim 6, wherein the computer caused to determine the plurality of concentrations further includes the computer caused to determine a next lowermost one of the concentrations that is immediately between the lowermost one of the plurality of concentrations and a second next lowermost one of the concentrations, and
wherein the next lowermost one of the concentrations is determined from division of the midpoint by an exponentiation of the target titration factor when the estimated IC₅₀ value is less than an upper concentration limit, and as the value that is equidistant between the lowermost and the second next lowermost ones of the plurality of concentrations when the estimated IC₅₀ value is greater than an upper concentration limit.

10. The system of claim 1, wherein the CRC is constructed as a second sigmoidal curve to which the plurality of concentrations and the plurality of response measurements are fit using the Hill equation.

11. The system of claim 10, wherein the Hill equation is a four-parameter logistic nonlinear model with parameters of the sigmoidal curve including a minimum asymptote, a maximum asymptote, an inflection point that corresponds to the IC₅₀, and a Hill slope, and
wherein the apparatus caused to construct the CRC includes the apparatus caused to perform a regression analysis of the four-parameter logistic nonlinear model in which the minimum asymptote, the maximum asymptote, the Hill slope and the inflection point are all unconstrained.

12. The system of claim 1, wherein the system further comprises laboratory equipment including a dispenser and at least one sensor, and the three concentrations are tested in a laboratory in which the dispenser is controllable to dispense the three concentrations into wells of a first assay plate, and the at least one sensor is configured to obtain the three response measurements are obtained, and
wherein the plurality of concentrations are tested in the laboratory with the laboratory equipment in which the dispenser is controllable to dispense the plurality of concentrations into the wells of a second assay plate, and the plurality of response measurements are obtained from the at least one sensor.

13. A method of screening a compound relative to a target biological component, the method comprising:
determining an estimated half maximal inhibitory concentration (IC₅₀) value for the compound relative to the target biological component from three concentrations of the compound, including:
testing the three concentrations on the target biological component to obtain three response measurements of the compound in inhibiting a biological function of the target;
constructing a pseudo concentration-response curve (CRC) from the three concentrations and the three response measurements, the pseudo CRC constructed as a sigmoidal curve to which the three concentrations and the three response measurements are fit using the Hill equation; and
determining the estimated IC₅₀ value from the pseudo CRC;
determining a plurality of concentrations of the compound from the estimated IC₅₀ value, the plurality of concentrations thereby specific to the compound relative to the target;
testing the plurality of concentrations of the compound on the target biological component to obtain a plurality of response measurements of the compound; and
constructing a CRC from the plurality of concentrations and the plurality of response measurements.

14. The method of claim 13, wherein the three concentrations have a dilution factor that is a defined multiplier for the three concentrations, and the three concentrations include a lowermost concentration, an intermediate concentration that is a multiple of the lowermost concentration for the defined multiplier, and an uppermost concentration that is a multiple of the intermediate concentration for the defined multiplier.

15. The method of claim 13, wherein the Hill equation is a four-parameter logistic nonlinear model with parameters of the sigmoidal curve including a minimum asymptote, a maximum asymptote, an inflection point that corresponds to the IC₅₀, and a Hill slope, and
wherein constructing the pseudo CRC includes performing a regression analysis of the four-parameter logistic nonlinear model in which the minimum asymptote, the maximum asymptote and the Hill slope are constrained to predetermined values, and the inflection point is unconstrained.

16. The method of claim 13, wherein determining the estimated IC₅₀ value includes determining an inflection point of the pseudo CRC that corresponds to the estimated IC₅₀ value.

17. The method of claim 13, wherein the plurality of concentrations of the compound are tested using a dispenser to dispense the plurality of concentrations, and uppermost and lowermost ones of the plurality of concentrations are determined based on maximum and minimum concentrations the dispenser is designed to dispense.

18. The method of claim 17, wherein the plurality of concentrations includes concentrations that are between uppermost and lowermost ones of the plurality of concentrations, and determining the plurality of concentrations includes:
defining a target titration factor as a dilution multiplier to achieve a defined multiplier across the concentrations, the defined multiplier indicated by a dilution factor for the three concentrations;
defining upper and lower IC₅₀ limits based on the estimated IC₅₀ value, and the target titration factor; and
determining a midpoint one of the concentrations that is the estimated IC₅₀ value when the estimated IC₅₀ value is between the upper and lower IC₅₀ limits, and a value that is equidistant between the upper and lower IC₅₀ limits when the estimated IC₅₀ value is not between the upper and lower IC₅₀ limits.

19. The method of claim 18, wherein determining the plurality of concentrations further includes determining those of the concentrations above the midpoint from multiplication of the midpoint and exponentiations of the target titration factor by respective exponents.

20. The method of claim 18, wherein determining the plurality of concentrations further includes determining at least some of those of the concentrations below the midpoint from division of the midpoint by exponentiations of the target titration factor by respective exponents.

21. The method of claim 18, wherein determining the plurality of concentrations further includes determining a next lowermost one of the concentrations that is immediately between the lowermost one of the plurality of concentrations and a second next lowermost one of the concentrations, and
wherein the next lowermost one of the concentrations is determined from division of the midpoint by an exponentiation of the target titration factor when the estimated IC₅₀ value is less than an upper concentration limit, and as the value that is equidistant between the lowermost and the second next lowermost ones of the plurality of concentrations when the estimated IC₅₀ value is greater than an upper concentration limit.

22. The method of claim 13, wherein the CRC is constructed as a second sigmoidal curve to which the plurality of concentrations and the plurality of response measurements are fit using the Hill equation.

23. The method of claim 22, wherein the Hill equation is a four-parameter logistic nonlinear model with parameters of the sigmoidal curve including a minimum asymptote, a maximum asymptote, an inflection point that corresponds to the IC₅₀, and a Hill slope, and
wherein constructing the CRC includes performing a regression analysis of the four-parameter logistic nonlinear model in which the minimum asymptote, the maximum asymptote, the Hill slope and the inflection point are all unconstrained.

24. The method of claim 13, wherein the three concentrations are tested in a laboratory with laboratory equipment including a dispenser controlled to dispense the three concentrations into wells of a first assay plate, and at least one sensor from which the three response measurements are obtained, and
wherein the plurality of concentrations are tested in the laboratory with the laboratory equipment in which the dispenser is controlled to dispense the plurality of concentrations into the wells of a second assay plate, and the plurality of response measurements are obtained from the at least one sensor.
